(19) United States
(12) Patent Application Publication
(10) Pub. No.: US 2023/0157667 A1
(43) Pub. Date: May 25, 2023

(54) FLEXIBLE SUBSTRATE WITH RECESSES FOR INTRALUMINAL ULTRASOUND IMAGING DEVICES

(71) Applicant: PHILIPS IMAGE GUIDED THERAPY CORPORATION, SAN DIEGO, CA (US)

(72) Inventors: Maritess MINAS, SAN DIEGO, CA (US); David Kenneth WROLSTAD, FALLBROOK, CA (US); Nathan Andrew WILLIAMS, SAN DIEGO, CA (US)

(21) Appl. No.: 17/908,549

(22) PCT Filed: Feb. 20, 2021
(86) PCT No.: PCT/EP2021/054246

§ 371 Date: Sep. 1, 2022

Related U.S. Application Data
(60) Provisional application No. 62/985,454, filed on Mar. 5, 2020.

(51) Int. Cl. A61B 8/00; A61B 8/12; A61B 8/08

(52) U.S. Cl. CPC A61B 8/445; A61B 8/12; A61B 8/0891; A61B 8/4488

(57) ABSTRACT

An intraluminal imaging catheter includes a flexible elongate member configured to be positioned within a body lumen of a patient. The flexible elongate member includes a proximal portion and a distal portion. The catheter includes an ultrasound imaging assembly coupled to the flexible elongate member at the distal portion. The ultrasound imaging assembly includes a flexible substrate. The flexible substrate includes a first surface and an opposite, second surface. The imaging assembly also includes an ultrasound transducer array disposed on the flexible substrate. The flexible substrate includes a first recess extending from the first surface to the second surface. The ultrasound imaging assembly is coupled to the flexible elongate member via a first adhesive positioned in a space between the flexible substrate and the flexible elongate member via the first recess.

### TECHNICAL FIELD

The present disclosure relates generally to intraluminal medical imaging and, in particular, to the distal structure of an intraluminal imaging device. For example, an intravascular ultrasound (IVUS) imaging catheter has a flexible substrate with recesses that allow adhesive penetration for coupling to other components with increased tensile strength.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

Solid-state (also known as synthetic-aperture) IVUS catheters are one of the two types of IVUS devices commonly used today, the other type being the rotational IVUS catheter. Solid-state IVUS catheters carry a scanner assembly that includes an array of ultrasound transducers distributed around its circumference along with one or more integrated circuit controller chips mounted adjacent to the transducer array. The controllers select individual acoustic elements (or groups of elements) for transmitting an ultrasound pulse and for receiving the ultrasound echo signal. By stepping through a sequence of transmit-receive pairs, the solid-state IVUS system can synthesize the effect of a mechanically scanned ultrasound transducer but without moving parts (hence the solid-state designation). Since there is no rotating mechanical element, the transducer array can be placed in direct contact with the blood and vessel tissue with minimal risk of vessel trauma. Furthermore, because there is no rotating element, the electrical interface is simplified. The solid-state scanner can be wired directly to the imaging system with a simple electrical cable and a standard detachable electrical connector, rather than the complex rotating electrical interface required for a rotational IVUS device.

Manufacturing solid-state IVUS devices that can efficiently traverse anatomic structures within the human body is challenging. IVUS devices must be extremely narrow to successfully pass through the human vasculature without damaging tissue. Despite their extremely small size, intraluminal imaging devices must also have high tensile strength to ensure that the device or parts of the device do not separate during a procedure. Such a breakage may cause parts of an intraluminal imaging device to be left within the heart or vasculature. Connections between various components of intraluminal imaging devices provide generally weaker tensile strength and are more prone to separation of other components of intraluminal imaging devices. In addition, current methods of connecting components of intraluminal imaging devices often require increased overall diameters of the device which may limit the ability of the device to maneuver through a patient's vasculature. An increased diameter at connections may also make the device less smooth and more disposed to agitate or damage tissues within the body.

### SUMMARY

Embodiments of the present disclosure are directed to connections of an intraluminal imaging device, such intravascular ultrasound (IVUS) catheter, at a distal and proximal end that overcome the limitations described above. For example, an IVUS imaging assembly is attached at a proximal end to electrical wires that transmit imaging data to and from a control and processing system and other elongate structures. The IVUS imaging assembly is also attached to at a distal end to a tip member of the catheter. The IVUS imaging assembly has a flexible substrate on which the ultrasound transducer elements are positioned. The flexible substrate also has multiple recesses (e.g., two or more recesses at the distal end of the flexible substrate and two or more recesses at the proximal end of the flexible substrate). The proximal end of the tip member has a smaller diameter than the distal end of the flexible substrate. During assembly of the catheter, a gap is created between the two components when the proximal end of the tip member and the distal end of the flexible substrate in its rolled form are brought together. The recesses in the flexible substrate are positioned over the gap. Adhesive is injected into the gap through one of the recesses to bond the two components. The second recess serves as a vent through which any air within the gap may escape. Similarly, two recesses at the proximal end of the flexible substrate may be positioned over a similar gap between the flexible substrate and an inner and outer catheter shaft. Adhesive passes through one recess and air passes through the other like a vent. This connection method results in increased tensile strength of bonds between components of the IVUS catheter and ensures a smaller overall diameter.

In an exemplary aspect, an intraluminal imaging catheter is provided. The intraluminal imaging catheter includes a flexible elongate member configured to be positioned within a body lumen of a patient, the flexible elongate member comprising a proximal portion and a distal portion; an ultrasound imaging assembly coupled to the flexible elongate member at the distal portion, wherein the ultrasound imaging assembly comprises: a flexible substrate comprising a first surface and an opposite, second surface; and an ultrasound transducer array disposed on the flexible substrate, wherein the flexible substrate comprises a first recess extending from the first surface to the second surface, and wherein the ultrasound imaging assembly is coupled to the flexible elongate member via a first adhesive positioned in a space between the flexible substrate and the flexible elongate member via the first recess.

In some aspects, the flexible substrate comprises a second recess extending from the first surface to the second surface, and the second recess is configured to vent air within the space when the first adhesive is positioned in the space between the flexible substrate and the flexible elongate member via the first recess. In some aspects, the flexible substrate comprises a proximal portion and a distal portion, and the first recess and second recess are disposed at the proximal portion of the flexible substrate. In some aspects, the flexible substrate comprises a rolled configuration, and the first recess and the second recess are disposed on opposite sides of the ultrasound imaging assembly when the flexible substrate is in the rolled configuration. In some aspects, the intraluminal imaging catheter further includes a tip member coupled to the ultrasound imaging assembly, the flexible substrate comprises a third recess extending from the first surface to the second surface, and the tip member is coupled to the ultrasound imaging assembly via a second adhesive positioned in a space between the flexible substrate and the tip member via the third recess. In some aspects, the flexible substrate comprises a fourth recess extending from the first surface to the second surface, and the fourth recess is configured to vent air within the space when the second adhesive is positioned in the space between the flexible substrate and the tip member via the third recess. In some aspects, the flexible substrate comprises a proximal portion and a distal portion, and the third recess and fourth recess are disposed at the distal portion of the flexible substrate. In some aspects, the ultrasound imaging assembly further comprises a support member, the flexible substrate is disposed around the support member, and the first adhesive is in contact with the support member, the flexible substrate, and the flexible elongate member. In some aspects, the flexible elongate member comprises an inner member and an outer member disposed around the inner member, and the first adhesive is positioned in the space between flexible substrate and at least one of the inner member or the outer member.

In an exemplary aspect, an intraluminal imaging catheter is provided. The intraluminal imaging catheter includes a flexible elongate member configured to be positioned within a body lumen of a patient, the flexible elongate member comprising a proximal portion and a distal portion; an ultrasound imaging assembly disposed at the distal portion of the flexible elongate member; and a tip member coupled to the ultrasound imaging assembly, wherein the ultrasound imaging assembly comprises: a flexible substrate comprising a first surface and an opposite, second surface; and an ultrasound transducer array disposed on the flexible substrate, wherein the flexible substrate comprises a first recess extending from the first surface to the second surface, and wherein the ultrasound imaging assembly is coupled to the tip member via a first adhesive positioned in a space between the flexible substrate and the tip member via the first recess.

In some aspects, the flexible substrate comprises a second recess extending from the first surface to the second surface, and the second recess is configured to vent air within the space when the first adhesive is positioned in the space between the flexible substrate and the tip member via the first recess. In some aspects, the flexible substrate comprises a proximal portion and a distal portion, and the first recess and second recess are disposed at the distal portion of the flexible substrate. In some aspects, the flexible substrate comprises a rolled configuration, and the first recess and the second recess are disposed on opposite sides of the ultrasound imaging assembly when the flexible substrate is in the rolled configuration. In some aspects, the flexible elongate member is coupled to the ultrasound imaging assembly, the flexible substrate comprises a third recess extending from the first surface to the second surface, and the flexible elongate member is coupled to the ultrasound imaging assembly via a second adhesive positioned in a space between the flexible substrate and the flexible elongate member via the third recess. In some aspects, the flexible substrate comprises a fourth recess extending from the first surface to the second surface, and the fourth recess is configured to vent air within the space when the second adhesive is positioned in the space between the flexible substrate and the flexible elongate member via the third recess. In some aspects, wherein the flexible substrate comprises a proximal portion and a distal portion, and the third recess and fourth recess are disposed at the proximal portion of the flexible substrate. In some aspects, the ultrasound imaging assembly further comprises a support member, the flexible substrate is disposed around the support member, and the first adhesive is in contact with the support member, the flexible substrate, and the tip member. In some aspects, an outer surface of the tip member comprises a first taper and an opposite, second taper, and the space between the flexible substrate and the tip member comprises a space between the first taper and the flexible elongate member.

In an exemplary aspect, an intravascular ultrasound (IVUS) imaging catheter is provided. The IVUS imaging catheter includes a flexible elongate member configured to be positioned within a body lumen of a patient, the flexible elongate member comprising a proximal portion and a distal portion; an ultrasound imaging assembly comprising a proximal portion and a distal portion; and a tip member coupled to the distal portion of the ultrasound imaging assembly, wherein the flexible elongate member is coupled to the proximal portion of the ultrasound imaging assembly, wherein the ultrasound imaging assembly comprises: a flexible substrate comprising a first surface and an opposite, second surface; and an ultrasound transducer array disposed on the flexible substrate, wherein the flexible substrate comprises a first recess, a second recess, a third recess, and a fourth recess each extending from the first surface to the second surface, wherein the first recess and the second recess are disposed at the proximal portion of the ultrasound imaging assembly, wherein the third recess and the fourth recess at the distal portion of the ultrasound imaging assembly, and wherein the ultrasound imaging assembly is coupled to the flexible elongate member via a first adhesive positioned in a space between the flexible substrate and the flexible elongate via the first recess while air is vented out of the second recess, and wherein the ultrasound imaging assembly is coupled to the tip member via a second adhesive positioned in a space between the flexible substrate and the tip member via the third recess while air is vented out of the fourth recess such that the flexible substrate defines an outer profile of the IVUS imaging catheter without the first or the second adhesive forming a larger profile than the outer profile.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an intraluminal imaging system, according to aspects of the present disclosure.

FIG. 2 is a diagrammatic perspective view of the top of a scanner assembly in a flat configuration, according to aspects of the present disclosure.

FIG. 3 is a diagrammatic perspective view of the scanner assembly shown in FIG. 2 in a rolled configuration around a support member, according to aspects of the present disclosure.

FIG. 4 is a diagrammatic cross-sectional side view of the scanner assembly shown in FIG. 3, according to aspects of the present disclosure.

FIG. 5 is a top view of a scanner assembly in a flat configuration, according to aspects of the present disclosure.

FIG. 6 is a diagrammatic cross-sectional view of the proximal connection between the scanner assembly, the support member, the inner member, and/or the outer member before adhesive is applied, according to aspects of the present disclosure.

FIG. 7 is a diagrammatic cross-sectional view of the proximal connection between the scanner assembly, the support member, the inner member, and/or the outer member after adhesive is applied, according to aspects of the present disclosure.

FIG. 8 is a diagrammatic cross-sectional view of the distal connection between the scanner assembly and the tip member before adhesive is applied, according to aspects of the present disclosure.

FIG. 9 is a diagrammatic cross-sectional view of the distal connection between the scanner assembly and the tip member after adhesive is applied, according to aspects of the present disclosure.

FIG. 10 is a diagrammatic top view of another embodiment of the scanner assembly in a flat configuration, according to aspects of the present disclosure.

FIG. 11 is a diagrammatic top view of another embodiment of the scanner assembly in a flat configuration, according to aspects of the present disclosure.

FIG. 12 is a diagrammatic top view of another embodiment of the scanner assembly in a flat configuration, according to aspects of the present disclosure.

FIG. 13 is a flow diagram of a method of assembling an intraluminal imaging device according to an embodiment of the present disclosure.

FIG. 14 is a side view of the scanner assembly shown in FIG. 5 in rolled configuration, positioned around a support member, the support member supported by an assembly mandrel, according to aspects of the present disclosure.

FIG. 15 is a side view of the scanner assembly, support member, and assembly mandrel shown in FIG. 14 with an inner member passing through the center of the scanner assembly and support member, according to aspects of the present disclosure.

FIG. 16 is a side view of the scanner assembly, support member, assembly mandrel, and inner member shown in FIG. 15 with an outer member positioned over the proximal leg of the flexible substrate and inner member, according to aspects of the present disclosure.

FIG. 17 is a side view of the scanner assembly, support member, assembly mandrel, inner member, and outer member shown in FIG. 16 with a tip member coupled to the distal end of the scanner assembly, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the focusing system is described in terms of cardiovascular imaging, it is understood that it is not intended to be limited to this application. The system is equally well suited to any application requiring imaging within a confined cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

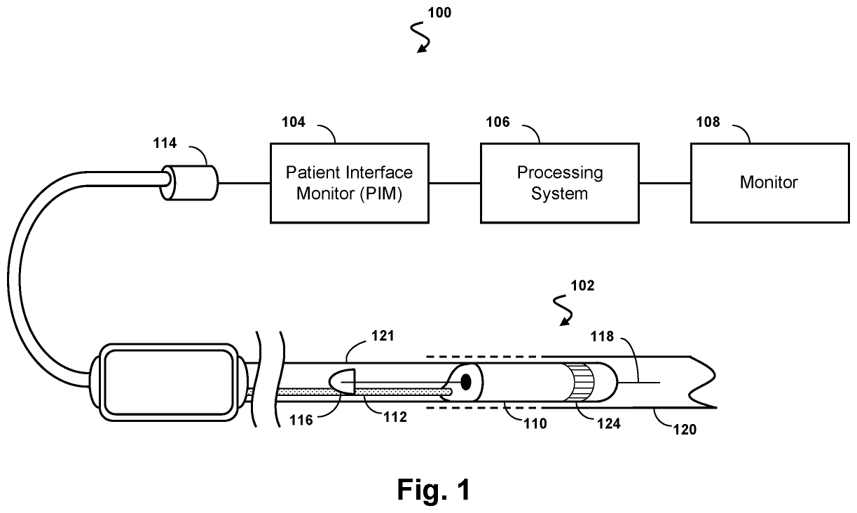

FIG. 1 is a diagrammatic schematic view of an intraluminal imaging system 100, according to aspects of the present disclosure. The intraluminal imaging system 100 can be an ultrasound imaging system. In some instances, the system 100 can be an intravascular ultrasound (IVUS) imaging system. The system 100 may include an intraluminal imaging device 102 such as a catheter, guide wire, or guide catheter, a patient interface module (PIM) 104, an processing system or console 106, and a monitor 108. The intraluminal imaging device 102 can be an ultrasound imaging device. In some instances, the device 102 can be an IVUS imaging device, such as a solid-state IVUS device. The intraluminal imaging device 102 may also be referred to as an intraluminal imaging catheter. The intraluminal imaging device may also be referred to as an intravascular ultrasound (IVUS) imaging catheter.

At a high level, the IVUS device 102 emits ultrasonic energy from a transducer array 124 included in scanner assembly 110 mounted near a distal end of the catheter device. The ultrasonic energy is reflected by tissue structures in the medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the IVUS imaging system 100 described herein. For example, the processor can execute computer readable instructions stored on the non-transitory tangible computer readable medium.

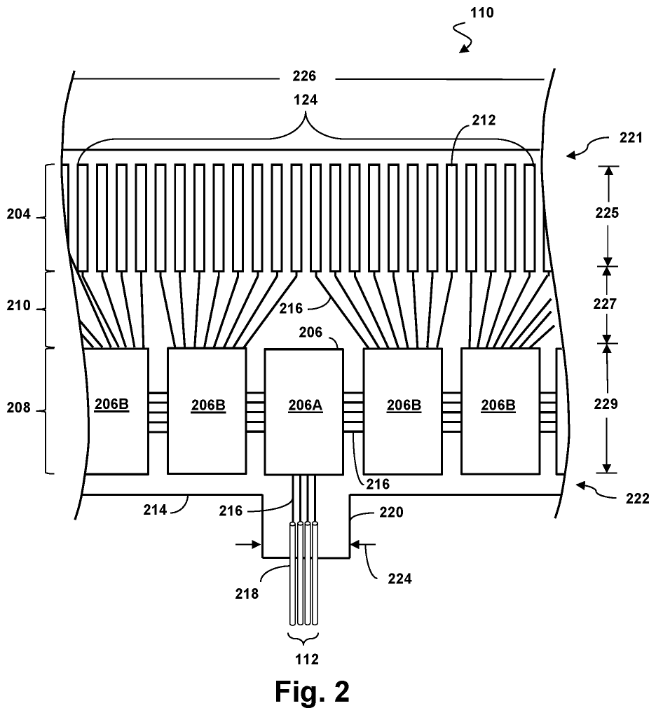

The PIM 104 facilitates communication of signals between the IVUS console 106 and the scanner assembly 110 included in the IVUS device 102. This communication includes the steps of: (1) providing commands to integrated circuit controller chip(s) 206A and 206B, illustrated in FIG. 2, included in the scanner assembly 110 to select the particular transducer array element(s), or acoustic element(s), to be used for transmit and receive, (2) providing the transmit trigger signals to the integrated circuit controller chip(s) 206A and 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s)126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The IVUS console 106 receives the echo data from the scanner assembly 110 by way of the PIM 104 and processes the data to reconstruct an image of the tissue structures in the medium surrounding the scanner assembly 110. The console 106 outputs image data such that an image of the vessel 120, such as a cross-sectional image of the vessel 120, is displayed on the monitor 108. Vessel 120 may represent fluid filled or surrounded structures, both natural and man-made. The vessel 120 may be within a body of a patient. The vessel 120 may be a blood vessel, as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the transmission line bundle or cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

FIG. 2 is a diagrammatic top view of a portion of a flexible assembly 110, according to aspects of the present disclosure. The flexible assembly 110 includes a transducer array 124 formed in a transducer region 204 and transducer control logic dies or controllers 206 (including dies 206A and 206B) formed in a control region 208, with a transition region 210 disposed therebetween. The transducer array 124 includes an array of ultrasound transducers 212. The transducer control logic dies 206 are mounted on a flexible substrate 214 into which the transducers 212 have been previously integrated. The flexible substrate 214 is shown in a flat configuration in FIG. 2. Though six control logic dies 206 are shown in FIG. 2, any number of control logic dies 206 may be used. For example, one, two, three, four, five, six, seven, eight, nine, ten, or more control logic dies 206 may be used.

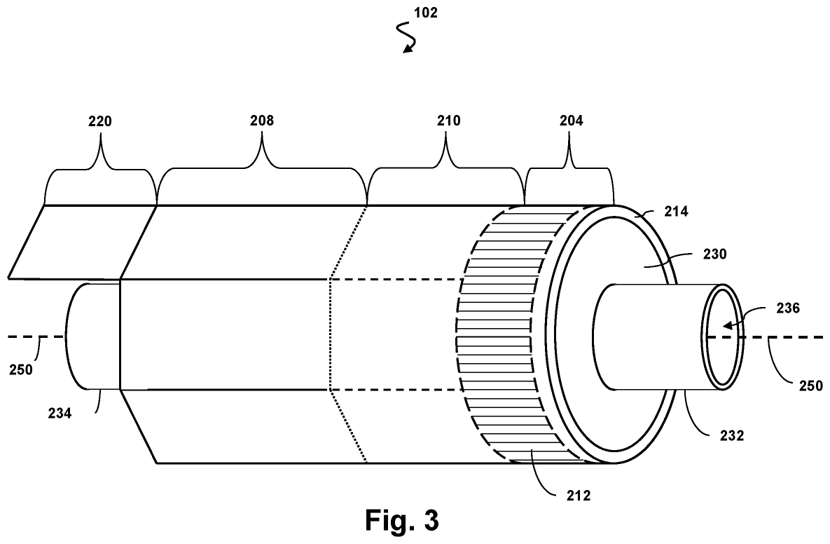

The flexible substrate 214, on which the transducer control logic dies 206 and the transducers 212 are mounted, provides structural support and interconnects for electrical coupling. The flexible substrate 214 may be constructed to include a film layer of a flexible polyimide material such as KAPTON™ (trademark of DuPont). Other suitable materials include polyester films, polyimide films, polyethylene napthalate films, or polyetherimide films, liquid crystal polymer, other flexible printed semiconductor substrates as well as products such as Upilex® (registered trademark of Ube Industries) and TEFLON® (registered trademark of E.I. du Pont). In the flat configuration illustrated in FIG. 2, the flexible substrate 214 has a generally rectangular shape. As shown and described herein, the flexible substrate 214 is configured to be wrapped around a support member 230 (FIG. 3) in some instances. Therefore, the thickness of the film layer of the flexible substrate 214 is generally related to the degree of curvature in the final assembled flexible assembly 110. In some embodiments, the film layer is between 5 μm and 100 μm, with some particular embodiments being between 5 μm and 25.1 μm, e.g., 6 μm.

The set of transducer control logic dies 206 is a non-limiting example of a control circuit. The transducer region 204 is disposed at a distal portion 221 of the flexible substrate 214. The control region 208 is disposed at a proximal portion 222 of the flexible substrate 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or, the length 227 of the transition region 210 may be less than lengths 225 and 229, the length 227 of the transition region 210 can be greater than lengths 225, 229 of the transducer region and controller region, respectively.

The control logic dies 206 are not necessarily homogenous. In some embodiments, a single controller is designated a master control logic die 206A and contains the communication interface for cable 112, between a processing system, e.g., processing system 106, and the flexible assembly 110. Accordingly, the master control circuit may include control logic that decodes control signals received over the cable 112, transmits control responses over the cable 112, amplifies echo signals, and/or transmits the echo signals over the cable 112. The remaining controllers are slave controllers 206B. The slave controllers 206B may include control logic that drives a transducer 212 to emit an ultrasonic signal and selects a transducer 212 to receive an echo. In the depicted embodiment, the master controller 206A does not directly control any transducers 212. In other embodiments, the master controller 206A drives the same number of transducers 212 as the slave controllers 206B or drives a reduced set of transducers 212 as compared to the slave controllers 206B. In an exemplary embodiment, a single master controller 206A and eight slave controllers 206B are provided with eight transducers assigned to each slave controller 206B.

To electrically interconnect the control logic dies 206 and the transducers 212, in an embodiment, the flexible substrate 214 includes conductive traces 216 formed in the film layer that carry signals between the control logic dies 206 and the transducers 212. In particular, the conductive traces 216 providing communication between the control logic dies 206 and the transducers 212 extend along the flexible substrate 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 112 when the conductors 218 of the cable 112 are mechanically and electrically coupled to the flexible substrate 214. Suitable materials for the conductive traces 216 include copper, gold, aluminum, silver, tantalum, nickel, and tin, and may be deposited on the flexible substrate 214 by processes such as sputtering, plating, and etching. In an embodiment, the flexible substrate 214 includes a chromium adhesion layer. The width and thickness of the conductive traces 216 are selected to provide proper conductivity and resilience when the flexible substrate 214 is rolled. In that regard, an exemplary range for the thickness of a conductive trace 216 and/or conductive pad is between 1-5 μm. For example, in an embodiment, 5 μm conductive traces 216 are separated by 5 μm of space. The width of a conductive trace 216 on the flexible substrate may be further determined by the width of the conductor 218 to be coupled to the trace/pad.

The flexible substrate 214 can include a conductor interface 220 in some embodiments. The conductor interface 220 can be in a location of the flexible substrate 214 where the conductors 218 of the cable 112 are coupled to the flexible substrate 214. For example, the bare conductors of the cable 112 are electrically coupled to the flexible substrate 214 at the conductor interface 220. The conductor interface 220 can be tab extending from the main body of flexible substrate 214. In that regard, the main body of the flexible substrate 214 can refer collectively to the transducer region 204, controller region 208, and the transition region 210. In the illustrated embodiment, the conductor interface 220 extends from the proximal portion 222 of the flexible substrate 214. In other embodiments, the conductor interface 220 is positioned at other parts of the flexible substrate 214, such as the distal portion 221, or the flexible substrate 214 may lack the conductor interface 220. A value of a dimension of the tab or conductor interface 220, such as a width 224, can be less than the value of a dimension of the main body of the flexible substrate 214, such as a width 226. In some embodiments, the substrate forming the conductor interface 220 is made of the same material(s) and/or is similarly flexible as the flexible substrate 214. In other embodiments, the conductor interface 220 is made of different materials and/or is comparatively more rigid than the flexible substrate 214. For example, the conductor interface 220 can be made of a plastic, thermoplastic, polymer, hard polymer, etc., including polyoxymethylene (e.g., DELRIN®), polyether ether ketone (PEEK), nylon, Liquid Crystal Polymer (LCP), and/or other suitable materials.

FIG. 3 illustrates a perspective view of the device 102 with the scanner assembly 110 in a rolled configuration. In some instances, the imaging assembly 110 is transitioned from a flat configuration (FIG. 2) to a rolled or more cylindrical configuration (FIG. 3). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

In some embodiments, the transducer elements 212 and/or the controllers 206 can be positioned in an annular configuration, such as a circular configuration or in a polygon configuration, around a longitudinal axis 250 of a support member 230. It will be understood that the longitudinal axis 250 of the support member 230 may also be referred to as the longitudinal axis of the scanner assembly 110, the flexible elongate member 121, and/or the device 102. For example, a cross-sectional profile of the imaging assembly 110 at the transducer elements 212 and/or the controllers 206 can be a circle or a polygon. Any suitable annular polygon shape can be implemented, such as one based on the number of controllers/transducers, flexibility of the controllers/transducers, etc., including a pentagon, hexagon, heptagon, octagon, nonagon, decagon, etc. In some examples, the plurality of transducer controllers 206 may be used for controlling the plurality of ultrasound transducer elements 212 to obtain imaging data associated with the vessel 120.

The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, ('220 application) the entirety of which is hereby incorporated by reference herein. The support member 230 can be a ferrule having a distal flange or portion 232 and a proximal flange or portion 234. The support member 230 can be tubular in shape and define a lumen 236 extending longitudinally therethrough. The lumen 236 can be sized and shaped to receive the guide wire 118. The support member 230 can be manufactured using any suitable process. For example, the support member 230 can be machined and/or electrochemically machined or laser milled, such as by removing material from a blank to shape the support member 230, or molded, such as by an injection molding process.

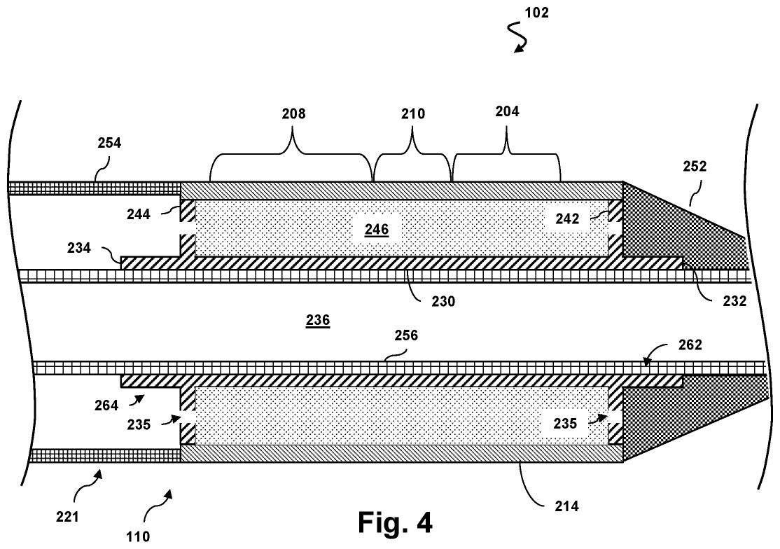

Referring now to FIG. 4, shown there is a diagrammatic cross-sectional side view of a distal portion of the intraluminal imaging device 102, including the flexible substrate 214 and the support member 230, according to aspects of the present disclosure. The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. The support member 230 can be ferrule having a distal portion 262 and a proximal portion 264. The support member 230 can define a lumen 236 extending along the longitudinal axis LA. The lumen 236 is in communication with the entry/exit port 116 and is sized and shaped to receive the guide wire 118 (FIG. 1). The support member 230 can be manufactured according to any suitable process. For example, the support member 230 can be machined and/or electrochemically machined or laser milled, such as by removing material from a blank to shape the support member 230, or molded, such as by an injection molding process. In some embodiments, the support member 230 may be integrally formed as a unitary structure, while in other embodiments the support member 230 may be formed of different components, such as a ferrule and stands 242, 244, that are fixedly coupled to one another. In some cases, the support member 230 and/or one or more components thereof may be completely integrated with inner member 256. In some cases, the inner member 256 and the support member 230 may be joined as one, e.g., in the case of a polymer support member.

Stands 242, 244 that extend vertically are provided at the distal and proximal portions 262, 264, respectively, of the support member 230. The stands 242, 244 elevate and support the distal and proximal portions of the flexible substrate 214. In that regard, portions of the flexible substrate 214, such as the transducer portion 204 (or transducer region 204), can be spaced from a central body portion of the support member 230 extending between the stands 242, 244. The stands 242, 244 can have the same outer diameter or different outer diameters. For example, the distal stand 242 can have a larger or smaller outer diameter than the proximal stand 244 and can also have special features for rotational alignment as well as control chip placement and connection. To improve acoustic performance, any cavities between the flexible substrate 214 and the surface of the support member 230 are filled with a liquid backing material 246. The liquid backing material 246 can be introduced between the flexible substrate 214 and the support member 230 via passageways 235 in the stands 242, 244. In some embodiments, suction can be applied via the passageways 235 of one of the stands 242, 244, while the liquid backing material 246 is fed between the flexible substrate 214 and the support member 230 via the passageways 235 of the other of the stands 242, 244. The backing material can be cured to allow it to solidify and set. In various embodiments, the support member 230 includes more than two stands 242, 244, only one of the stands 242, 244, or neither of the stands. In that regard the support member 230 can have an increased diameter distal portion 262 and/or increased diameter proximal portion 264 that is sized and shaped to elevate and support the distal and/or proximal portions of the flexible substrate 214.

The support member 230 can be substantially cylindrical in some embodiments. Other shapes of the support member 230 are also contemplated including geometrical, non-geometrical, symmetrical, non-symmetrical, cross-sectional profiles. As the term is used herein, the shape of the support member 230 may reference a cross-sectional profile of the support member 230. Different portions of the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

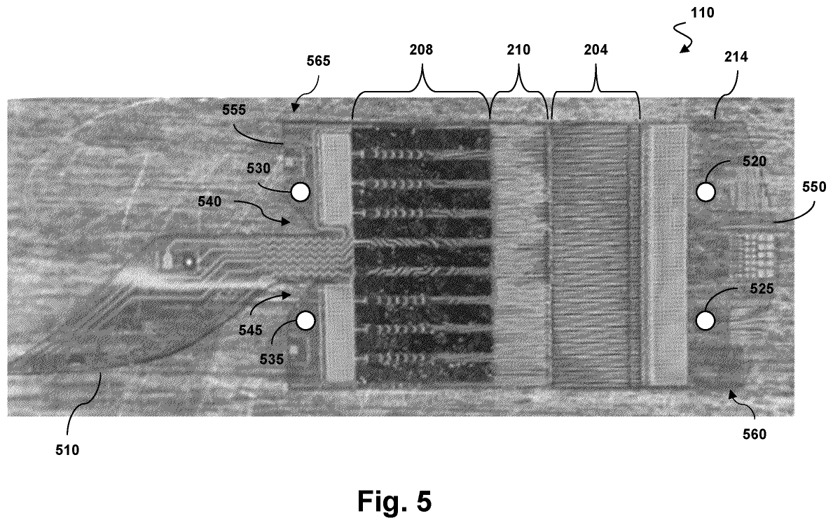

A proximal inner member 256 and a proximal outer member 254 are coupled to the proximal portion 264 of the support member 230. A flexible elongate member may comprise the inner member 256 and/or the proximal outer member 254. The proximal inner member 256 can be received within a proximal flange 234. The outer member 254 may abut and be in contact with the proximal end 555 (FIG. 5) of flexible substrate 214. In other embodiments, the outer member 254 may be positioned within the lumen created by the inner surface of flexible substrate 214 and the outer surface of support member 230. The outer surface of outer member 254 may be in contact with the inner surface of flexible substrate 214. A distal tip member 252 is coupled to the distal portion 262 of the support member 230. For example, the distal member 252 is positioned around the distal flange 232. The tip member 252 can abut and be in contact with the distal end 550 (FIG. 5) of flexible substrate 214 and the stand 242. In other embodiments, the proximal end of the tip member 252 may be received within the distal end 555 of the flexible substrate 214 in its rolled configuration. In some embodiments there may be a gap between the flexible substrate 214 and the tip member 252. The distal member 252 can be the distal-most component of the intraluminal imaging device 102.

One or more adhesives can be disposed between various components at the distal portion of the intraluminal imaging device 102. For example, one or more of the flexible substrate 214, the support member 230, the distal member 252, the proximal inner member 256, and/or the proximal outer member 254 can be coupled to one another via an adhesive.

FIG. 5 is a top view of the scanner assembly 110 in a flat configuration, according to aspects of the present disclosure. The scanner assembly 110 may include a flexible substrate 214 on which various components may be disposed. The flexible substrate 214 may include a distal end 550 and a proximal end 555. As previously mentioned, the flexible substrate may include control region 208, transducer region 204, and transition region 210 positioned therebetween. Flexible substrate 214 comprises a first or outer surface and a second or inner surface such that when the flexible substrate is in its rolled configuration, the first or outer surface is positioned radially outward and the second or inner surface is positioned radially inward creating a lumen.

Coupled to the proximal end 555 of the flexible substrate 214 may be a proximal leg 510. The proximal leg 510 may extend proximally to the flexible substrate 214 as shown in FIG. 5. The proximal leg 510 may be positioned along the center line of the scanner assembly 110 in its flat configuration or may be positioned in any other suitable location along the proximal end 555 of the flexible substrate 214. The proximal leg also need not extend exactly proximally from the scanner assembly 110 but may extend in any direction relative to the scanner assembly 110. The proximal leg 510 may extend toward one side of the center line of the scanner assembly 110 as shown in FIG. 5 such that the proximal leg wraps in a spiral manner around the outer surface of the outer member 254 and the inner member 256 when the scanner assembly 110 is in its rolled configuration. Conductive traces, other conductors, electrical components, integrated circuit controller chips, or various other suitable components may be disposed on the surface of the proximal leg 510. Proximal leg 510 may be used to mechanically and electrically couple the scanner assembly 110 to the transmission line bundle or cable 112. The proximal leg 510 may be constructed of the same material as the flexible substrate 214. For example, proximal leg 510 may be constructed of a flexible polyimide material or any other materials including polyester films, polyimide films, polyethylene napthalate films, or polyetherimide films, liquid crystal polymer, or any other flexible printed semiconductor substrates. The proximal leg 510 may be of any suitable length. The exact dimensions of the proximal leg 510 are selected to ensure a secure coupling between the proximal leg 510 and the flexible substrate 214 and between the proximal leg 510 and the transmission line bundle or cable 112. The dimensions of the proximal leg 510 may also be selected to ensure that the intraluminal imaging device 102 is sufficiently narrow and flexible to successfully maneuver through the vasculature of a patient. The intraluminal imaging device 102 may include features substantially similar to those described in International Publication No. WO 2017/168300, titled “Imaging Assembly for Intravascular Imaging Device and Associated Devices, Systems, and Methods,” and U.S. Application No. 62/789,099, titled “INCREASED FLEXIBILITY SUBSTRATE FOR INTRALUMINAL ULTRASOUND IMAGING ASSEMBLY,” and filed Jan. 7, 2019 (Atty. Dkt. No. 2018PF00854/44755.1986PV01), each of which is hereby incorporated by reference in its entirety.

The proximal end 555 of the flexible substrate 214 and the proximal leg 510 may also be configured with notches 540 and 545 disposed on either side of proximal leg 510 as shown in FIG. 5. Notches 540 and 545 may be configured to receive an additional component such as outer member 254, or various other components of similar shape and dimension, while the scanner assembly 110 is in its rolled configuration. In its rolled configuration, the scanner assembly 110 may receive outer member 254 such that the distal end of the outer member 254 is received into notches 540 and 545 in such a way that the proximal leg 510 is positioned within the inner lumen of outer member 254 but the proximal region 565 is positioned around the outer surface of outer member 254. When received into notches 540 and 545 of flexible substrate 214, outer member 254 may abut the flexible substrate 214 at various locations along the edge of notches 540 and 545.

A plurality of holes or recesses may be positioned within substrate 214. As shown in FIG. 5, a first recess 520 and a second recess 525 are positioned within the distal region 560 of flexible substrate 214. Although two recesses 520 and 525 are depicted in FIG. 5, any suitable number of at least two may be positioned within the distal region 560 of flexible substrate 214, including, three, four, or more. Recesses 520 and 525 may be positioned within flexible substrate 214 in such a way that when the scanner assembly 110 is in its rolled configuration, recesses 520 and 525 are then positioned at generally opposite sides of the rolled scanner assembly 110. In other embodiments, recesses 520 and 525 may be positioned in different locations along flexible substrate 214. For example, recess 520 may be positioned substantially 90 degrees in a circumferential or azimuthal direction from recess 525 when scanner assembly 110 is in its rolled configuration. In other embodiments, recess 520 may be positioned further or closer to recess 525 depending on the specific application. In addition, recesses 520 and 525 need not be positioned at the same position longitudinally as is depicted in FIG. 5. For example, either recess 520 or 525 may be positioned further distally or proximally to one another. Recesses 520 and 525 extend completely through flexible substrate 214 such that recesses 520 and 525 extend from the first or outer surface of flexible substrate 214 to the second or inner surface of flexible substrate 214. As discussed in more detail hereafter, recesses 520 and 525 may serve respectively as an inlet through which adhesive may be injected within the scanner assembly 110 in its rolled configuration and as a vent through which air within the scanner assembly 110 may escape during an adhesive injection process. Recess 520 may serve as an inlet and recess 525 may serve as a vent or vice versa. Any additional recesses introduced into the design of scanner assembly 110 may function in similar fashion as inlets or vents or may serve other purposes. The dimensions of recesses 520 and 525 may be selected according to the overall dimensions of the scanner assembly 110, the viscosity or other characteristics of adhesive used, or various other parameters. For example, a minimum diameter of recesses 520 and 525 may be between 0.1″ and 0.2″. However, this diameter is merely exemplary, and the scanner assembly 110 and corresponding recesses 520 and 525 may be of any suitable dimension depending on the specific application (e.g., cardiac vasculature, peripheral vasculature, etc.).

A third recess 530 and a fourth recess 535 are positioned at the proximal region 565 of the flexible substrate 214 and may be configured in a substantially similar way to recesses 520 and 525. Additional recesses may be positioned at or near the proximal region 565 of the flexible substrate 214. For example, recesses 530 and 535 may be positioned within flexible substrate 214 in such a way that when the scanner assembly 110 is in its rolled configuration, recesses 530 and 535 are then positioned at generally opposite sides of the rolled scanner assembly 110. Recess 530 and recess 535 may be disposed on opposite sides of the intraluminal imaging catheter or intraluminal imaging device 102 when the flexible substrate 214 is in its rolled configuration. Recess 520 and recess 525 may also be disposed on opposite sides of the intraluminal imaging catheter or intraluminal imaging device 102. Recesses 530 and 535 may also be positioned in different circumferential or longitudinal directions from one another, similar to embodiments of recesses 520 and 525 as previously discussed. Similar to recesses 520 and 525, recesses 530 and 535 extend completely through flexible substrate 214 such that recesses 530 and 535 extend from the first or outer surface of flexible substrate 214 to the second or inner surface of flexible substrate 214. Recesses 530 and 535 may serve as an inlet through which adhesive may be injected and/or otherwise provided within the scanner assembly 110 in its rolled configuration and as a vent through which air within the scanner assembly 110 may escape during an adhesive injection process respectively or vice versa. Additional recesses may also be included within the proximal region 565 at various other locations to serve as adhesive inlets or air vents. Recesses 530 and 535 may be of substantially similar dimensions as recesses 520 and 525, or may be substantially different. As previously noted in regard to recesses 520 and 525, the dimensions of recesses 530 and 535 may be of any suitable dimension depending on the specific application.

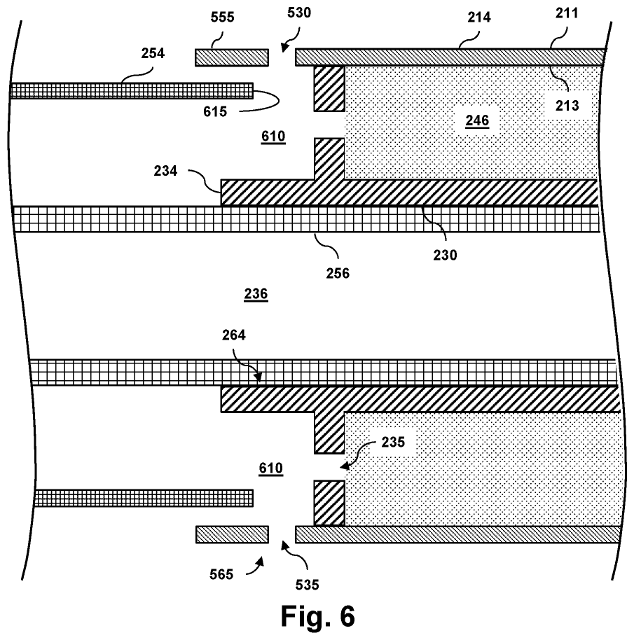

Recesses 520, 525, 530, and 535 may extend completely through flexible substrate 214. For example, flexible substrate 214 comprises an upper surface, outer surface, or first surface 211 (FIG. 6) and a lower surface or second surface 213 (FIG. 6). Recesses 520, 525, 530, and 535 extend from upper surface 211 of flexible substrate 214 completely through flexible substrate 214 to lower surface, inner surface, or second surface 213 of flexible substrate 214. In this manner, the lumen created by flexible substrate 214 when it is in its rolled configuration is in direct communication with the environment (e.g., radially inward and radially outward) surrounding flexible substrate 214 by way of recesses 520, 525, 530, and 535.

FIG. 6 is a diagrammatic cross-sectional view of the proximal connection between the flexible substrate 214, the support member 230, the inner member 256, and/or the outer member 254 before adhesive is applied, according to aspects of the present disclosure. As shown in FIG. 6, the outer member 254 is positioned around the inner member 256. In some embodiments, the overall diameter of the outer member 254 may be of a smaller diameter than the overall diameter of the proximal end 555 of the flexible substrate 214. In such an embodiment, distal end 615 of the outer member 254 may be received within the proximal end 555 of the flexible substrate 214. The distal end 615 of outer member 254 may be received within cavity or lumen 610 created between the inner surface of flexible substrate 214 and the outer surface of support member 230. Distal end 615 of outer member 254 may extend any distance within cavity 610. In other embodiments, the distal end 615 of the outer member 254 may abut the proximal end 555 of the flexible substrate 214. The distal end 615 of the outer member 254 may abut the proximal end 555 of the flexible substrate 214 at a location distal to the proximal end 234 of the support member 230. In other embodiments, the distal end 615 of the outer member 254 may abut the proximal end 555 of the flexible substrate 214 at a location proximal to the proximal end 234 of the support member 230 or at the same general location of the proximal end 234 of support member 230. In still other embodiments, the distal end 615 of the outer member 254 may not abut the proximal end 555 of the flexible substrate 214, but may leave a gap. Recesses 530 and 535 may be positioned on either side of scanner assembly 110. Recesses 530 and 535 may be positioned over a cavity 610 within the proximal region of scanner assembly 110. The cavity 610 may be located between the proximal end 234 of the support member 230, the proximal region 565 of the flexible substrate 214, and the distal end 615 of the outer member 254. Cavity 610 may extend azimuthally or circumferentially around the cylindrical body of the imaging assembly and is therefore depicted both above and below lumen 236 of the support member 230 in the longitudinal cross-sectional view of FIG. 6. To mechanically couple the distal end of the outer member 254 to the proximal end of the scanner assembly 110, one of either recesses 530 or 535 acts as an inlet and the other acts as a vent. Either recess 530 or recess 535 may be used as an inlet or a vent interchangeably, however, for the purposes of the present application, recess 530 will be described as an inlet, and recess 535 will be described as a vent. It is fully contemplated that recess 535 may be used as an inlet and recess 530 may be used as a vent. During the connection process, adhesive is injected and/or otherwise provided through the inlet recess 530. To allow the adhesive to flow through inlet recess 530 and fill cavity 610, vent recess 535 allows gases in cavity 610 to escape. Recess 530 may be configured to receive adhesive and recess 535 may be configured to vent air.

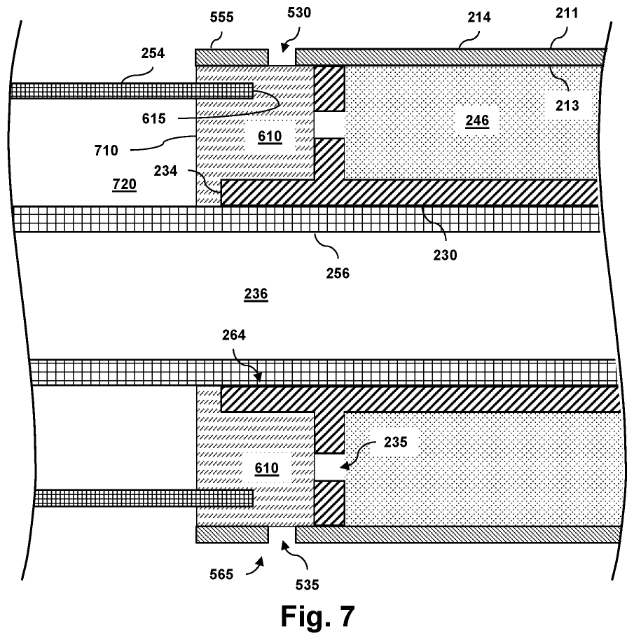

FIG. 7 is a diagrammatic cross-sectional view of the proximal connection between the flexible substrate 214, the support member 230, the inner member 256, and/or the outer member 254 after adhesive 710 is applied, according to aspects of the present disclosure. As shown in FIG. 7, after adhesive 710 is injected into the cavity 610, adhesive 710 comes in direct contact with the flexible substrate 214, the support member 230, the inner member 256, and/or the outer member 254 creating a strong mechanical coupling between these components. In addition, this method of connecting components of an ultrasound imaging assembly maintains the same overall diameter of the device at connection locations as shown in FIG. 7 such that the outer diameter of the flexible substrate 214 is the largest overall outer diameter of any component longitudinally of the intraluminal imaging device 102. This is due to the fact that adhesive 710 is positioned radially interior to the flexible substrate 214 through the use of recess 530 as an inlet and recess 535 as a vent, as opposed to using a fillet, or other method of connection surrounding the flexible substrate 214 and extending radially outward. In this manner, the flexible substrate 214 defines an outer profile of the IVUS imaging catheter 102 without the adhesive or other method of connection forming a larger profile than the outer profile. In some embodiments, adhesive 710 may flow proximally into cavity 720 defined as the region proximal to the scanner assembly 110 between the outer member 254 and the inner member 256. In other embodiments, the amount of adhesive 710 which may flow into cavity 720 may be controlled by the amount of adhesive 710 injected into cavity 610, the viscosity of adhesive 710, the orientation of scanner assembly 110 during the adhesive injection process, or a variety of other factors. In still other embodiments, a barrier may be included with the scanner assembly 110 between cavity 720 and cavity 610, or positioned at any other suitable location, which may prevent adhesive 710 from flowing proximally into cavity 720. This barrier may be a separate component. It may also be a part of or connected to outer member 254, a part of or connected to flexible substrate 214, a part of or connected to support member 230, or a part of or connected to inner member 256. In other embodiments, a part of adhesive 710 may additionally flow along the exterior surface of outer member 254 in a longitudinally proximal direction. The amount of adhesive 710 which may flow over the exterior surface of outer member 254 may similarly be controlled by the amount of adhesive 710 injected, the viscosity of adhesive 710 and other previously mentioned factors. Physically barriers may also be placed as part of or connected to outer member 254 or as part of or connected to the flexible substrate 214 to restrict the flow of adhesive 710 over the exterior surface of outer member 254. In some embodiments, the distal end 615 of outer member 254 may be completely surrounded by and adhered to adhesive 710, such that a portion of the outer surface of outer member 254 comes in direct contact with adhesive 710 and a portion of the inner surface of outer member 254 comes in direct contact with adhesive 710. In other embodiments, only an inner surface of outer member 254 may be in contact with adhesive 710. In still other embodiments, only an outer surface of outer member 254 may be in contact with adhesive 710.

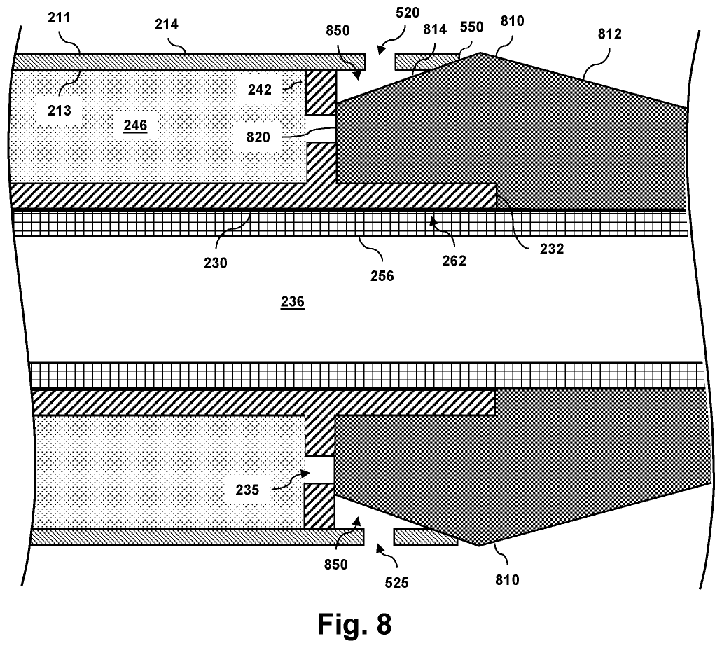

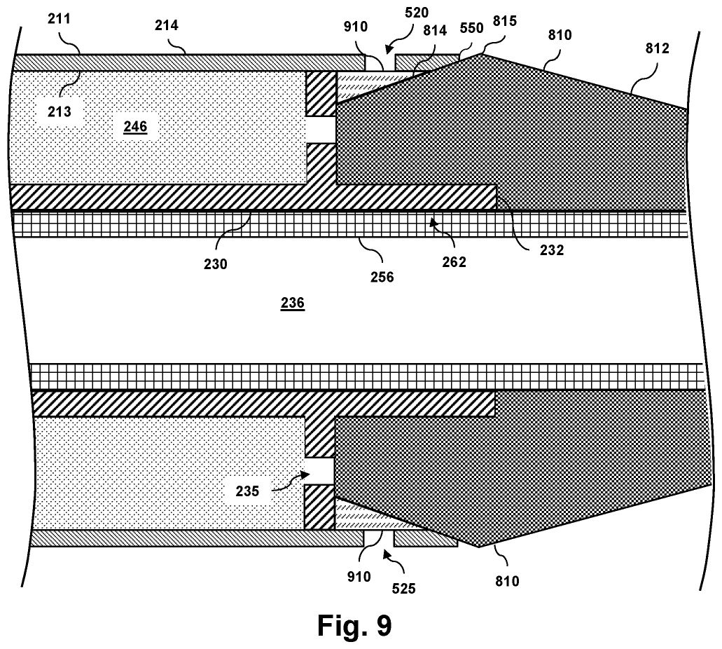

FIG. 8 is a diagrammatic cross-sectional view of the distal connection between the scanner assembly 110 and a tip member 810 before adhesive is applied, according to aspects of the present disclosure. The tip member 810 may substantially similar to distal tip member 252 of FIG. 4, or may differ substantially. Tip member 810 may be a generally conical shaped element with a small overall diameter at its distal end which gradually increases along slope or taper 812 to a point 815 of the same general diameter as the scanner assembly 110. At the proximal end of tip member 810, as shown in FIG. 9, the diameter of the tip member 810 may again gradually decrease along slope or taper 814 to a smaller diameter than that of the scanner assembly 110. This taper 814 of the proximal end of the tip member 810 allows the proximal end 820 of the tip member 810 to be inserted into the distal end of the flexible substrate 214 in its rolled configuration. In some embodiments, the proximal end 820 of the tip member 810 may be inserted into the lumen created by the flexible substrate 214 in its rolled configuration until it abuts the stand 242 of the support member 230 or other parts of support member 230. In other embodiments, the proximal end 820 of the tip member 810 may not abut stand 242 of support member 230 but may be positioned at some point distal of stand 242 of support member 230.

The outer surface of tip member 810 may come in direct contact with the distal end 550 of flexible substrate 214. A gap 850 may then be created between the outer surface of tip member 810 along taper 814, the inner surface of flexible substrate 214, and the distal end of the stand 242 of support member 230, or other regions of support member 230. As with cavity 610 of the proximal connection between the scanner assembly 110 and the outer member 254, gap 850 may extend circumferentially around the cylindrical body of the imaging assembly and is therefore depicted both above and below the lumen 236 of the support member 230 in the longitudinal cross-sectional view of FIG. 8. To mechanically couple the proximal end of the tip member 810 to the distal end of the scanner assembly 110, one of either recess 520 or 555, depicted in FIG. 8, acts as an inlet and the other acts as a vent. Either recess 520 or recess 525 may be used as either an inlet or a vent interchangeably, however, for the purposes of the present application only, recess 520 will be described as an inlet, and recess 525 will be described as a vent. During the connection process, adhesive is injected and/or otherwise provided through the inlet recess 520. To allow the adhesive to flow through inlet recess 520 and fill gap 850, vent recess 525 allows gases in gap 850 to escape. Recess 520 may be configured to receive adhesive and recess 525 may be configured to vent air.

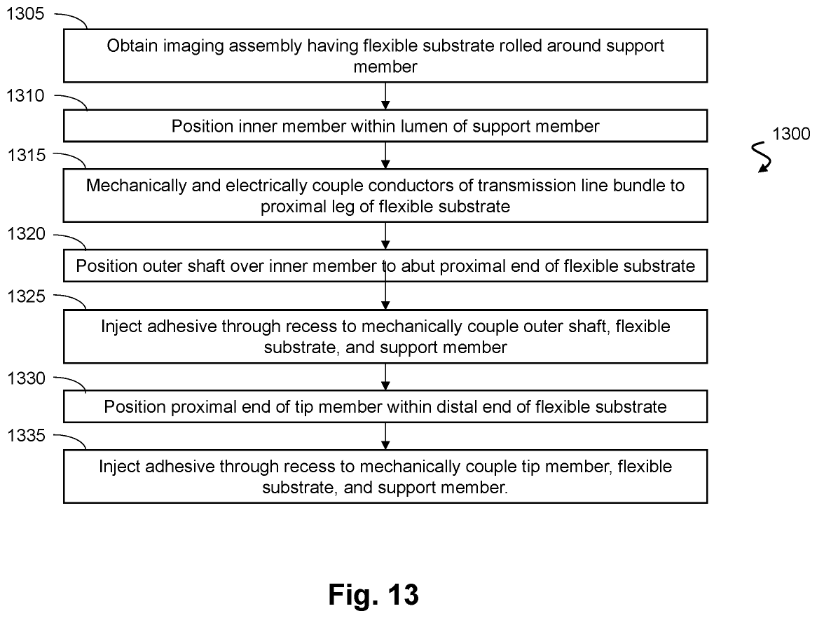

FIG. 9 is a diagrammatic cross-sectional view of the distal connection between the scanner assembly 110 and the tip member 810 after adhesive 910 is applied, according to aspects of the present disclosure. As shown in FIG. 9, after adhesive 910 is injected into the gap 850, adhesive 910 comes in direct contact with the flexible substrate 214, the stand 242 of the support member 230 or other region of support member 230, and the tip member 810 creating a strong mechanical coupling between these components. In addition, this method of connecting components of an ultrasound imaging assembly maintains the same overall diameter of the device at connection locations as shown in FIG. 13. As previously mentioned, this is due to the fact that adhesive 910 is positioned radially interior to the flexible substrate 214 through the use of recess 520 as an inlet and recess 525 as a vent, as opposed to using a fillet, or other method of connection surrounding the flexible substrate 214 and extending radially outward. In some embodiments, gap 850 and subsequently adhesive 910 may come in contact directly with support member 230 rather than stand 242 of support member 230. Adhesive 910 may be any particular type of suitable adhesive, such as epoxy, cyanoacrylate, urethane adhesive, and/or acrylic adhesives, as well as others. Adhesive 910 may be liquid of any suitable viscosity.

The tip member 810 depicted in FIGS. 8, 9, and 17 is merely illustrative and can be of various sizes or shapes and may be constructed of various materials. For example, tip member 810 may be constructed of a polymer, silicone rubber, nylon, polyurethane, polyethylene terephthalate (PET), latex, or other suitable materials. Further, tip member 810 may have general dimensions similar to those of the scanner assembly 110 or may be substantially larger or smaller than scanner assembly 110.

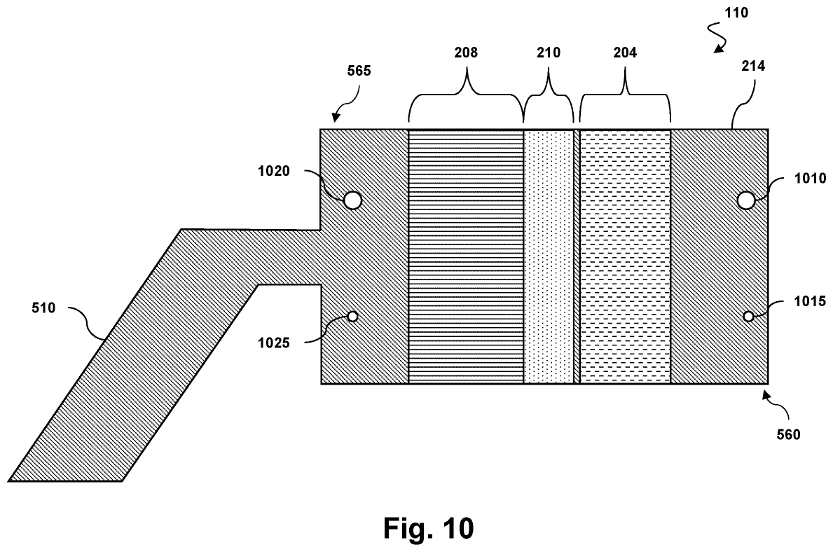

FIG. 10 is a diagrammatic top view of another embodiment of the scanner assembly 110 in a flat configuration, according to aspects of the present disclosure. At the distal region 560 of the flexible substrate 214, two recesses, recess 1010 and recess 1015 are depicted. These recesses may be substantially similar to recesses 520 and 525 of previous figures. However, recess 1015 is of a smaller diameter than recess 1010. Despite the difference in size, recess 1010 may serve as an inlet and recess 1015 may serve as a vent or vice versa. It is fully contemplated that additional recesses of various different sizes may also be introduced in a design as additional inlets or vents. Similarly, recesses 1020 and 1025 are positioned in the proximal region 565 of flexible substrate 214. Recesses 1020 and 1025 may be substantially similar to recesses 530 and 535 of previous figures. However, recess 1025 is of a smaller diameter than recess 1020. Again, either recess may serve as an inlet or a vent as previously described.

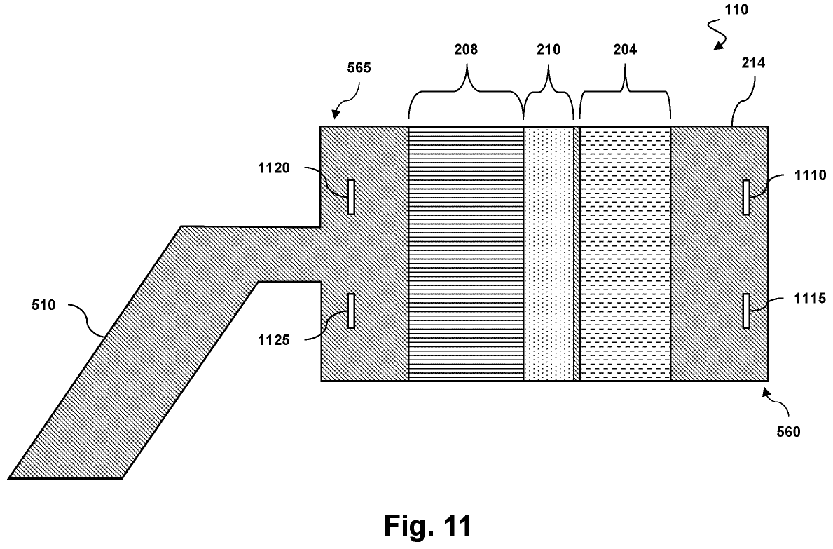

FIG. 11 is a diagrammatic top view of another embodiment of the scanner assembly 110 in a flat configuration, according to aspects of the present disclosure. At the distal region 560 of the flexible substrate 214, two recesses, recess 1110 and recess 1115 are depicted. These recesses may be substantially similar to recesses 520 and 525 of previous figures. However, recesses 1110 and 1115 are of a rectangular shape, rather than a circular shape as previously discussed. Despite the difference in shape, the recesses may still serve the same purpose as an inlet and vent or vice versa. It is fully contemplated that additional recesses of various different shapes may also be introduced in a design as additional inlets or vents. These shapes may include circles, rectangles, ovals, triangles, polygons, and other shapes. Similarly, recesses 1120 and 1125 are positioned in the proximal region 565 of flexible substrate 214. Recesses 1120 and 1125 may be substantially similar to recesses 530 and 535 of previous figures. However, recesses 1120 and 1125 are also of a rectangular shape. Again, either recess may serve as an inlet or a vent as previously described and different shapes of all types are fully contemplated.

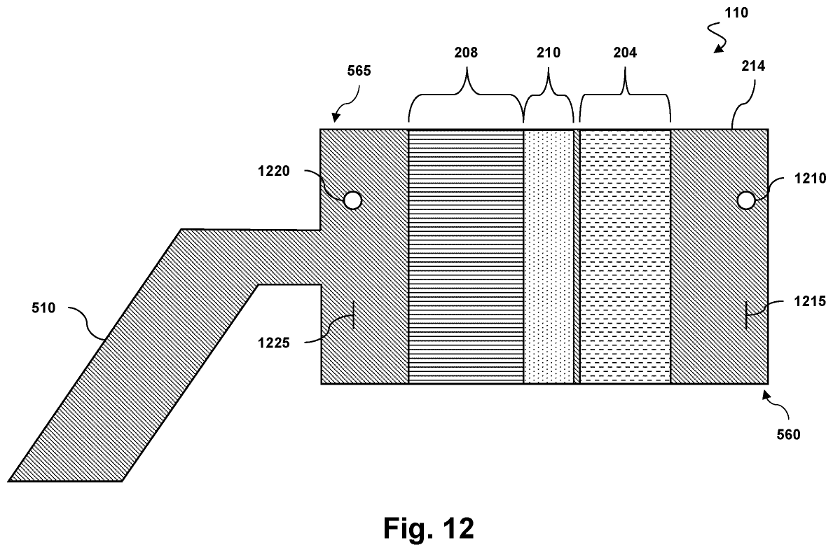

FIG. 12 is a diagrammatic top view of another embodiment of the scanner assembly in a flat configuration, according to aspects of the present disclosure. At the distal region 560 of the flexible substrate 214, a recess 1210 and a slit 1215 are depicted. Recess 1210 may be substantially similar to recess 520 of previous figures. However, a slit 1215 replaces previously presented recesses. Similarly, recess 1210 in some embodiments may similarly be a slit. Despite the difference in shape, the recesses or slits may still serve the same purpose as an inlet and vent or vice versa. It is fully contemplated that additional recesses or slits may also be introduced in a design as additional inlets or vents. Similarly, recess 1220 and slit 1225 are positioned in the proximal region 565 of flexible substrate 214. Recess 1220 may be substantially similar to recess 530 of previous figures. However, slit 1225 replaces previously described recesses. Again, either recess 1220 or slit 1225 may serve as an inlet or a vent as previously described and different shapes or configurations of perforations in flexible substrate 214 of all types are fully contemplated.

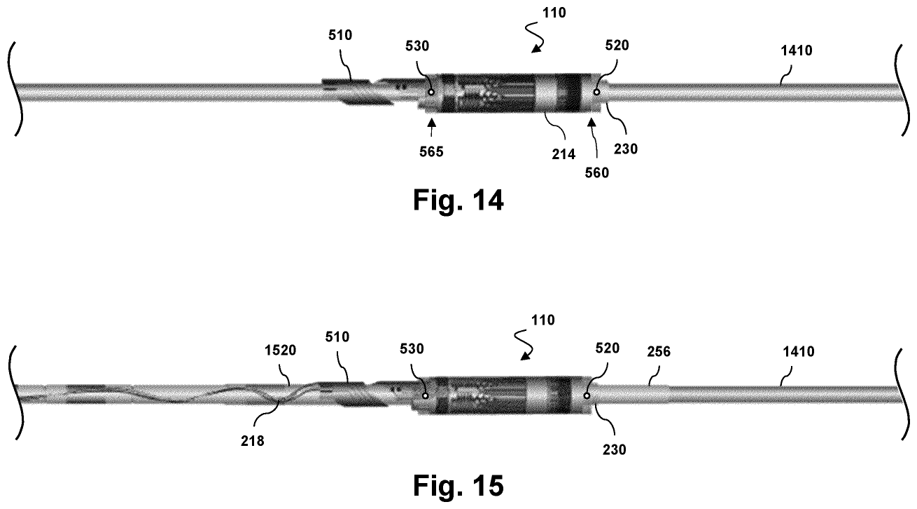

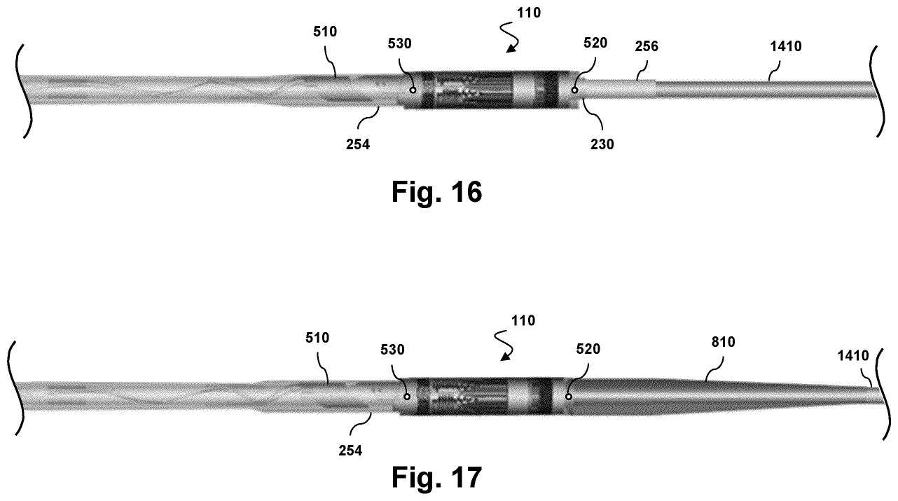

FIG. 13 is a flow chart diagram of a method 1300 of assembling an intraluminal imaging device 102 according to an embodiment of the present disclosure. The method 1300 can include mechanically coupling outer member 254 to the flexible substrate 214 and support member 230, and mechanically coupling tip member 810 to flexible substrate 214 and support member 230. As illustrated, method 1300 includes a number of enumerated steps, but embodiments of method 1300 may include additional steps before, after, or in between the enumerated steps. In some embodiments, one or more of the enumerated steps may be omitted, performed in a different order, or performed concurrently. The steps of method 1300 can be carried out by a manufacturer of the intraluminal imaging device 102, a manufacturer of a subassembly including the scanner assembly 110, the outer member 254, the tip member 810, and/or a manufacturer of any other component discussed in the present disclosure. Method 1300 will be described with reference to FIGS. 14-17, which are side views of various components of the ultrasound imaging assembly 102 during various steps of manufacturing. For example, FIGS. 14-17 illustrate assembly steps for various components of the ultrasound imaging assembly 102, such as the connection between the scanner assembly 110 and outer member 254 and the connection between the scanner assembly 110 and the tip member 810.

At step 1305, method 1300 includes obtaining an imaging assembly 102 having flexible substrate 214 rolled around support member 230. Step 1305 of obtaining an imaging assembly 102 may comprise a subprocess of manufacturing or assembling the imaging assembly 102 including positioning support member 230 on assembly mandrel 1410 and wrapping flexible substrate 214 around support member 230 and coming in contact with stands 242 and 244 of support member 230. An assembly mandrel 1410 may be used to support the ultrasound imaging assembly 102 during various stages of manufacturing. Assembly mandrel 1410 may be of any suitable length. The diameter of assembly mandrel 1410 may correspond to the inner diameter of the inner member 256 or may differ. In other embodiments, ultrasound imaging assembly 102 may be constructed without the use of assembly mandrel 1410. FIG. 14 is a side view of the scanner assembly 110 similar to that shown in FIG. 5 in rolled configuration during a stage of the assembly process. Specifically, in FIG. 14, scanner assembly 110 is depicted positioned around the support member 230. The support member 230 is further supported by the assembly mandrel 1410 as previously stated, according to aspects of the present disclosure. The proximal leg 510 is also depicted proximal to the scanner assembly 110 and wrapped in a spiral manner around the proximal portion of the assembly mandrel 1410. Recesses 520 and 530 are also depicted at the distal region 560 and proximal region 565 of the flexible substrate 214 respectively. Recesses 525 and 535 are not depicted as they are positioned on the opposite side of the scanner assembly 110 in its rolled configuration in this particular embodiment. In other embodiments, recesses 525 and 535 may be visible. The ultrasound transducer array may be disposed in a circumferential arrangement around a longitudinal axis of the scanner assembly 110.

At step 1310, method 1300 includes positioning inner member 256 within lumen 236 of support member 230. FIG. 7 is a side view of the scanner assembly 110, support member 230, and assembly mandrel 1410 shown in FIG. 6 with the inner member 256 passing through the lumen 236 of the support member 230, according to aspects of the present disclosure. Inner member 256 can comprise a flexible elongate member. Inner member 256 may be a flexible elongate member constructed of a polymer material that defines a lumen for various other components to pass through. Inner member 256 may be constructed of any number of suitable materials including polyethylene, polypropylene, polystyrene, and other suitable materials that offer flexibility, resistance to corrosion, and lack of conductivity.

As further shown in FIG. 15, a strain relief layer 1520 may be positioned around inner member 256 near the scanner assembly 110. Strain relief layer 1520 may include some features similar to those disclosed in U.S. Application No. 62/789,184 titled “STRAIN RELIEF FOR INTRALUMINAL ULTRASOUND IMAGING AND ASSOCIATED DEVICES, SYSTEMS, AND METHODS,” and filed Jan. 7, 2019 (Atty. Dkt. No. 2018PF00451/44755.1946PV01), which is hereby incorporated by reference in its entirety.

At step 1315, method 1300 includes mechanically and electrically coupling conductors 218 of transmission line bundle or cable 112 to proximal leg 510 of flexible substrate 214. As also shown in FIG. 15, a plurality of conductors 218 may be mechanically and electrically coupled to the proximal leg 510 at this stage of assembly. However, in other embodiments, conductors 218 may be mechanically and electrically coupled to proximal leg 510 at any other stage of the manufacturing process, including before the flexible substrate 214 is rolled around support member 230, after tip member 810 is coupled to scanner assembly 110 or at any point therebetween. Conductors 218 may be housed together within the transmission line bundle or cable 112, or may be independently positioned. Conductors 218 may be positioned around the outer layer of inner member 256 and extend proximally from scanner assembly 110 between scanner assembly 110 and PIM 104 or may be positioned in alternative locations along the scanner assembly 110 or inner member 256. As previously discussed, control signals and echo or imaging data may be transmitted and received over the conductors 218.

At step 1320, method 1300 includes positioning outer member 254 over the outer surfaces of inner member 256 and strain relief layer 1520 to abut the proximal end 555 of the flexible substrate 214. FIG. 8 is a side view of the scanner assembly 110, support member 230, assembly mandrel 1410, and inner member 256 with an outer member 254 positioned over the proximal leg 510 and inner member 256, according to aspects of the present disclosure. In some embodiments, and as previously shown in FIG. 6, the distal end 615 of outer member 254 may abut the proximal end 555 of flexible substrate 214. However, in other embodiments, the distal end 615 may be situated beneath the proximal end 555 of flexible substrate 214 in such a way that the distal region of outer member 254 overlaps with the proximal region of flexible substrate 214. In still other embodiments, the distal end 615 of outer member 254 may be positioned over or around the outer surface 211 of flexible substrate 214 such that the same overlapping of components is achieved, however, in reverse order.

At step 1325, method 1300 includes injecting adhesive 710 through recess 530 to mechanically couple the outer member 254, the flexible substrate 214, the inner member 256 and/or the support member 230. As has been discussed in more detail previously, after the distal end of outer member 254 is positioned proximate to, adjacent to, abutting, and/or in contact with the proximal region 565 of flexible substrate 214 in its rolled configuration, a cavity or lumen 610 exists between the flexible substrate 214, the supporting member 230 and the outer member 254. Adhesive may be injected through recess 530 and air within the gap may escape through recess 535 allowing the adhesive to fill the gap and couple flexible substrate 214 and supporting member 230 to outer member 254 resulting in both superior strength of the proximal connection and a lower profile around the area.

At step 1330, method 1300 includes positioning the proximal end of tip member 810 within the lumen defined by the distal end of flexible substrate 214 in its rolled configuration and flange 232 of support member 230. FIG. 17 is a side view of the scanner assembly 110, support member 230, assembly mandrel 1410, inner member 256, and outer member 254 with a tip member 810 positioned within the distal region 560 of the flexible substrate 214, according to aspects of the present disclosure. As has been previously discussed, the proximal end of the tip member 810 may be of a lesser overall diameter than the diameter of the lumen created by the distal region 560 of the flexible substrate 214 in its rolled configuration, such that the proximal end of the tip member 810 may be inserted into the lumen at the distal end of flexible substrate 214. Recess 520 within the distal region 560 of flexible substrate 214 may then be positioned over the proximal region of tip member 810.

At step 1335, method 1300 includes injecting adhesive 910 into gap 850 to mechanically couple tip member 810, flexible substrate 214, and support member 230. Similar to the adhesive injection process described in relation to the proximal connection of the outer member 254 with flexible substrate 214, and discussed previously in more detail, a gap between the flexible substrate 214, the support member 230 and the tip member 810 may exist within the lumen of the distal region 560 of the flexible substrate 214. Subsequently, recess 520 may act as an inlet for adhesive and recess 525 may act as a vent for air within the gap to escape allowing the adhesive to fill the gap and couple flexible substrate 214 and supporting member 230 to tip member 810.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraluminal imaging catheter, comprising:
a flexible elongate member configured to be positioned within a body lumen of a patient, the flexible elongate member comprising a proximal portion and a distal portion;
an ultrasound imaging assembly coupled to the flexible elongate member at the distal portion, wherein the ultrasound imaging assembly comprises:
a flexible substrate comprising a first surface and an opposite, second surface; and
an ultrasound transducer array disposed on the flexible substrate,
wherein the flexible substrate comprises a first recess extending from the first surface to the second surface, and
wherein the ultrasound imaging assembly is coupled to the flexible elongate member via a first adhesive positioned in a space between the flexible substrate and the flexible elongate member via the first recess.

2. The intraluminal imaging catheter of claim 1, wherein the flexible substrate comprises a second recess extending from the first surface to the second surface, and wherein the second recess is configured to vent air within the space when the first adhesive is positioned in the space between the flexible substrate and the flexible elongate member via the first recess.

3. The intraluminal imaging catheter of claim 2, wherein the flexible substrate comprises a proximal portion and a distal portion, wherein the first recess and second recess are disposed at the proximal portion of the flexible substrate.

4. The intraluminal imaging catheter of claim 2, wherein the flexible substrate comprises a rolled configuration, and wherein the first recess and the second recess are disposed on opposite sides of the ultrasound imaging assembly when the flexible substrate is in the rolled configuration.

5. The intraluminal imaging catheter of claim 2, further comprising a tip member coupled to the ultrasound imaging assembly, wherein the flexible substrate comprises a third recess extending from the first surface to the second surface, and wherein the tip member is coupled to the ultrasound imaging assembly via a second adhesive positioned in a space between the flexible substrate and the tip member via the third recess.

6. The intraluminal imaging catheter of claim 5, wherein the flexible substrate comprises a fourth recess extending from the first surface to the second surface, and wherein the fourth recess is configured to vent air within the space when the second adhesive is positioned in the space between the flexible substrate and the tip member via the third recess.

7. The intraluminal imaging catheter of claim 6, wherein the flexible substrate comprises a proximal portion and a distal portion, wherein the third recess and fourth recess are disposed at the distal portion of the flexible substrate.

8. The intraluminal imaging catheter of claim 1, wherein the ultrasound imaging assembly further comprises a support member, wherein the flexible substrate is disposed around the support member, and wherein the first adhesive is in contact with the support member, the flexible substrate, and the flexible elongate member.

9. The intraluminal imaging catheter of claim 1, wherein the flexible elongate member comprises an inner member and an outer member disposed around the inner member, wherein the first adhesive is positioned in the space between flexible substrate and at least one of the inner member or the outer member.

10. An intraluminal imaging catheter, comprising:
a flexible elongate member configured to be positioned within a body lumen of a patient, the flexible elongate member comprising a proximal portion and a distal portion;
an ultrasound imaging assembly disposed at the distal portion of the flexible elongate member; and
a tip member coupled to the ultrasound imaging assembly,
wherein the ultrasound imaging assembly comprises:
a flexible substrate comprising a first surface and an opposite, second surface; and
an ultrasound transducer array disposed on the flexible substrate,
wherein the flexible substrate comprises a first recess extending from the first surface to the second surface, and
wherein the ultrasound imaging assembly is coupled to the tip member via a first adhesive positioned in a space between the flexible substrate and the tip member via the first recess.

11. The intraluminal imaging catheter of claim 10, wherein the flexible substrate comprises a second recess extending from the first surface to the second surface, and wherein the second recess is configured to vent air within the space when the first adhesive is positioned in the space between the flexible substrate and the tip member via the first recess.

12. The intraluminal imaging catheter of claim 11, wherein the flexible substrate comprises a proximal portion and a distal portion, wherein the first recess and second recess are disposed at the distal portion of the flexible substrate.

13. The intraluminal imaging catheter of claim 11, wherein the flexible substrate comprises a rolled configuration, and wherein the first recess and the second recess are disposed on opposite sides of the ultrasound imaging assembly when the flexible substrate is in the rolled configuration.

14. The intraluminal imaging catheter of claim 11, wherein the flexible elongate member is coupled to the ultrasound imaging assembly, wherein the flexible substrate comprises a third recess extending from the first surface to the second surface, and wherein the flexible elongate member is coupled to the ultrasound imaging assembly via a second adhesive positioned in a space between the flexible substrate and the flexible elongate member via the third recess.

15. The intraluminal imaging catheter of claim 14, wherein the flexible substrate comprises a fourth recess extending from the first surface to the second surface, and wherein the fourth recess is configured to vent air within the space when the second adhesive is positioned in the space between the flexible substrate and the flexible elongate member via the third recess.

16. The intraluminal imaging catheter of claim 15, wherein the flexible substrate comprises a proximal portion and a distal portion, wherein the third recess and fourth recess are disposed at the proximal portion of the flexible substrate.

17. The intraluminal imaging catheter of claim 14, wherein the ultrasound imaging assembly further comprises a support member, wherein the flexible substrate is disposed around the support member, and wherein the first adhesive is in contact with the support member, the flexible substrate, and the tip member.

18. The intraluminal imaging catheter of claim 10, wherein an outer surface of the tip member comprises a first taper and an opposite, second taper, wherein the space between the flexible substrate and the tip member comprises a space between the first taper and the flexible elongate member.

19. An intravascular ultrasound (IVUS) imaging catheter, comprising:
a flexible elongate member configured to be positioned within a body lumen of a patient, the flexible elongate member comprising a proximal portion and a distal portion;
an ultrasound imaging assembly comprising a proximal portion and a distal portion; and
a tip member coupled to the distal portion of the ultrasound imaging assembly,
wherein the flexible elongate member is coupled to the proximal portion of the ultrasound imaging assembly,
wherein the ultrasound imaging assembly comprises:
a flexible substrate comprising a first surface and an opposite, second surface; and
an ultrasound transducer array disposed on the flexible substrate,
wherein the flexible substrate comprises a first recess, a second recess, a third recess, and a fourth recess each extending from the first surface to the second surface,
wherein the first recess and the second recess are disposed at the proximal portion of the ultrasound imaging assembly,
wherein the third recess and the fourth recess at the distal portion of the ultrasound imaging assembly, and
wherein the ultrasound imaging assembly is coupled to the flexible elongate member via a first adhesive positioned in a space between the flexible substrate and the flexible elongate via the first recess while air is vented out of the second recess, and wherein the ultrasound imaging assembly is coupled to the tip member via a second adhesive positioned in a space between the flexible substrate and the tip member via the third recess while air is vented out of the fourth recess such that the flexible substrate defines an outer profile of the IVUS imaging catheter without the first or the second adhesive forming a larger profile than the outer profile.
